(19) United States
(12) Patent Application Publication
(10) Pub. No.: US 2010/0016913 A1
(43) Pub. Date: Jan. 21, 2010

(54) INTERMITTENT PACING THERAPY FOR ANGINA AND DISEASE PREVENTION

(76) Inventors: Shantha Arcot-Krishnamurthy, Vadnais Heights, MN (US); Allan C. Shuros, St. Paul, MN (US); Robert Shipley, Austin, TX (US); Jason J. Hamann, Blaine, MN (US)

Correspondence Address: SCHWEGMAN, LUNDBERG & WOESSNER/BSC-CRM, PO BOX 2938, MINNEAPOLIS, MN, 55402, US

(21) Appl. No.: 12/504,602

(22) Filed: Jul. 16, 2009

Related U.S. Application Data
(60) Provisional application No. 61/081,272, filed on Jul. 16, 2008.

(51) Int. Cl. A61N 1/365

(52) U.S. Cl. 607/14; 607/17; 607/19

(57) ABSTRACT

A pacing system delivers cardiac protective pacing therapy (CPPT) to protect the heart from injuries and/or to treat existing injuries. The pacing system receives a set of inputs and delivers optimized cardiac protection pacing tailored for each of different purposes. The system delivers electrical stimulation to provide therapy for angina and/or to provide therapy for co-morbidities related to neural imbalance. In one embodiment, a method for treating angina is provided. A signal is sensed indicative of an incidence of angina and an angina region being a myocardial region affected by the angina. The incidence of angina is detected and the angina region is located. A pacing location is selected remote from the angina region, and CPPT is initiated at the pacing location. The CPPT is adapted to create increased stress at the angina region, to promote mass-redistribution and angiogenesis at the angina region to treat the angina.

### CROSS REFERENCE TO RELATED APPLICATIONS

This application claims the benefit of U.S. Provisional Application No. 61/081,272, filed on Jul. 16, 2008, under 35 U.S.C. § 119(e), which is hereby incorporated by reference.

This application is related to co-pending, commonly assigned, U.S. Patent Application No. 61/081,268, entitled “APPARATUS AND METHODS FOR TREATMENT OF ATHEROSCLEROSIS AND INFARCTION”, filed on Jul. 16,2008, which is hereby incorporated by reference in its entirety.

### TECHNICAL FIELD

This application relates generally to medical devices and, more particularly, to systems, devices and methods for implementing cardiac therapy.

### BACKGROUND

The heart is the center of a person's circulatory system. It includes an electro-mechanical system performing two major pumping functions. The left portions of the heart draw oxygenated blood from the lungs and pump it to the organs of the body to meet their metabolic requirements. The right portions of the heart draw deoxygenated blood from the body organs and pump it to the lungs where the blood gets oxygenated. These pumping functions are resulted mainly from contractions of the myocardium. In a normal heart, the sinoatrial node, the heart's natural pacemaker, generates electrical impulses that propagate through an electrical conduction system to various regions of the heart to excite the myocardial tissues of these regions. Coordinated delays in the propagations of the electrical impulses in a normal electrical conduction system cause the various portions of the heart to contract in synchrony resulting in an efficient pump. A blocked or otherwise abnormal electrical conduction and/or deteriorated myocardial tissue cause dysynchronous contraction of the heart, resulting in poor hemodynamic performance, including a diminished blood supply to the heart and the rest of the body. The condition where the heart fails to pump enough blood to meet the body's metabolic needs is known as heart failure.

Myocardial infarction (MI) is the necrosis of the myocardial tissue resulted from cardiac ischemia, a condition in which the myocardium is deprived of adequate oxygen and metabolite removal due to an interruption in blood supply caused by an occlusion of a blood vessel such as a coronary artery. The necrotic tissue, known as infarcted tissue, loses the contractile properties of the normal, healthy myocardial tissue. Consequently, the overall contractility of the myocardium is weakened, resulting in an impaired hemodynamic performance. Following an MI, cardiac remodeling starts with expansion of the region of infarcted tissue and progresses to a chronic, global expansion in the size and change in the shape of the entire left ventricle. The consequences include a further impaired hemodynamic performance and a significantly increased risk of developing heart failure, as well as a risk of suffering recurrent MI.

Heart disease such as MI and/or heart failure can cause adverse ventricular remodeling and an imbalance in autonomic tone favoring sympathetic activity over parasympathetic tone. During heart disease, the compromised ventricles may be less than capable of maintaining normal cardiac output. As a result, the body compensates for the reduced cardiac output by increasing sympathetic tone and suppressing parasympathetic activity, resulting in increased heart rate, myocardial contractility and blood volume. This mechanism is acutely beneficial, but has a long-term deleterious effect.

It has been shown experimentally that stressors delivered intermittently, such as exercise, dobutamine infusion, myocardial pacing, or external counterpulsation provide beneficial conditioning effects for the heart and body. Intermittent stress (e.g. exercise) improved the imbalance in the autonomic tone, as the autonomic tone trended from a predominantly sympathetic tendency toward a desired autonomic balance between the sympathetic and parasympathetic systems. For example, intensive exercise training in patients with reduced ventricular function has been shown to result in a significant improvement in exercise capacity (increased O₂ uptake, maximum minute ventilation, CO₂ production, exercise time and watts, a reduced baseline heart rate indicative of increased parasympathetic influence), with no deleterious effects on left ventricular volume, function or wall thickness. A potential mechanism for the benefit may be that these short intervals of stress increase sympathetic tone and cause a reflexive increase in parasympathetic tone after the stress is discontinued. Many HF and post-MI patients, however, are either debilitated and cannot exercise or do not tolerate exercise well enough to exercise effectively.

Intermittent sympathomimetic stimulation in animals with dobutamine produces benefits analogous to those of physical conditioning. In a pilot clinical study, patients with stable moderate severe HF (EF=23%) who received dobutamine therapy (30 min/day, 4 days/week, 3 weeks) experienced the following benefits: increased exercise tolerance; improved heart rate variability; lowered peripheral vascular resistance; and reduced plasma noradrenaline.

It has been proposed to deliver intermittent stress in the form of artificial cardiac pacing as a potential therapy for cardiac disease. A patient may not experience the desired benefit if the pacing delivers too little stress, or may be harmed (similar to over-exercising) if the pacing delivers too much stress.

Atherosclerosis begins with the appearance of cholesterol-laden macrophages (foam cells) in the intima of an artery. Smooth muscle cells respond to the presence of lipid by proliferating, under the influence of platelet factors. A plaque forms at the site, consisting of smooth muscle cells, leukocytes, and further deposition of lipid; in time the plaque becomes fibrotic and may calcify. Expansion of an atherosclerotic plaque leads to gradually increasing obstruction of the artery and ischemia of tissues supplied by it. Ulceration, thrombosis, or embolization of a plaque, or intimal hemorrhage and dissection, can cause more acute and severe impairment of blood flow, with the risk of infarction.

Treatment of atherosclerosis includes balloon stretching, laser ablation, or surgical removal of plaques, and various bypass and grafting procedures. Current preventive measures for atherosclerosis include regular vigorous exercise, a diet low in fat and cholesterol, maintenance of a healthful weight, avoidance of tobacco, and use of pharmacologic agents as indicated.

### SUMMARY

A pacing system delivers cardiac protective pacing therapy (CPPT) to protect the heart from injuries and/or to treat existing injuries. The pacing system receives a set of inputs and delivers optimized cardiac protection pacing tailored for different purposes. The system automatically adjusts heart rate to optimize cardiac protection pacing in a closed-loop system. The system delivers electrical stimulation to modulate myocardial strain for anti-atherosclerosis therapy, to provide therapy for myocardial infarction (MI), to provide therapy for angina, and/or to provide therapy for co-morbidities related to neural imbalance.

In one embodiment, a method for treating angina is provided. A signal is sensed indicative of an incidence of angina and an angina region being a myocardial region affected by the angina. The incidence of angina is detected and the angina region is located. A pacing location is selected remote from the angina region, and CPPT is initiated at the pacing location. The CPPT is adapted to create increased stress at the angina region, to promote mass-redistribution and angiogenesis at the angina region to treat the angina.

In one embodiment, a method for treating co-morbidities related to neural imbalance is provided. Cardiac protective pacing therapy (CPPT) is delivered to provide a cardiac conditioning therapy to improve neural balance. Neural balance and a parameter indicative of a selected co-morbidity related to neural imbalance are monitored, and the CPPT is titrated based on the monitored balance and parameter.

In one embodiment, a medical device for treating angina is provided. The device includes a sensing circuit to receive sensed signal indicative of an incidence of angina and an angina region being a myocardial region affected by the angina. The device also includes a pacemaker circuit adapted to deliver an electrical signal through at least one electrode to a pacing location remote from the angina region. According to various embodiments, the device further includes a controller to communicate with the sensing circuit and to control the pacemaker circuit to provide CPPT to the pacing location adapted to create increased stress at the angina region, to promote mass-redistribution and angiogenesis at the angina region to treat the angina.

In one embodiment, a medical device for treating co-morbidities related to neural imbalance is provided. The device includes a sensing circuit adapted to receive sensed signals indicative of neural balance and adapted to receive sensed parameters indicative of a selected co-morbidity related to neural imbalance. The device also includes a pacemaker circuit adapted to deliver an electrical signal through at least one electrode to a target location. The device further includes a controller adapted to communicate with the sensing circuit and to control the pacemaker circuit to provide CPPT to the target location, and further adapted to titrate the CPPT based on the sensed signals and parameters.

This Summary is an overview of some of the teachings of the present application and not intended to be an exclusive or exhaustive treatment of the present subject matter. Further details about the present subject matter are found in the detailed description and appended claims. Other aspects will be apparent to persons skilled in the art upon reading and understanding the following detailed description and viewing the drawings that form a part thereof, each of which are not to be taken in a limiting sense. The scope of the present invention is defined by the appended claims and their equivalents.

### BRIEF DESCRIPTION OF THE DRAWINGS

FIG. 1A illustrates the autonomic response to a period of exercise.

FIG. 1B illustrates the autonomic response to a period of cardiac protective pacing therapy (CPPT).

FIG. 2A is a flow chart illustrating an embodiment of a method for delivering pacing pulses for treating atherosclerosis.

FIG. 2B is a block diagram illustrating an embodiment of a medical device for delivering pacing pulses for treating atherosclerosis.

FIG. 3 illustrates the effect of pacing on strain patterns.

FIG. 4A is a flow chart illustrating a method for ventricular pacing to treat MI, according to various embodiments of the present subject matter.

FIG. 4B illustrates a method for pacing to treat MI, according to various embodiments of the present subject matter.

FIG. 4C illustrates electrode location for pacing to treat MI, according to an embodiment.

FIG. 5 is a flow chart illustrating a method for treating angina, according to an embodiment of the present subject matter.

FIG. 6 illustrates the effect of cardiac resynchronization therapy (CRT) on regional myocardial oxygen consumption.

FIG. 7 illustrates the effect of pacing on strain patterns relative to an angina region, according to various embodiments.

FIG. 8 is a flow chart illustrating an embodiment of a method for treating co-morbidities related to neural imbalance.

FIG. 9 is a flow chart illustrating an embodiment of a method using intermittent pacing to treat co-morbidities related to neural imbalance in a closed loop system.

FIG. 10 is an illustration of an embodiment of a cardiac rhythm management (CRM) system including an implantable system and an external system and portions of an environment in which the CRM system is used.

FIG. 11 is a block diagram illustrating an embodiment of portions of the circuit of a cardiac pacing system of the implantable system.

FIG. 12 is a block diagram illustrating an embodiment of portions of circuits of the implantable system and the external system.

FIG. 13 is a block diagram illustrating an embodiment of the external system.

### DETAILED DESCRIPTION

The following detailed description of the present subject matter refers to the accompanying drawings which show, by way of illustration, specific aspects and embodiments in which the present subject matter may be practiced. These embodiments are described in sufficient detail to enable those skilled in the art to practice the present subject matter. Other embodiments may be utilized and structural, logical, and electrical changes may be made without departing from the scope of the present subject matter. References to “an”, “one”, or “various” embodiments in this disclosure are not necessarily to the same embodiment, and such references contemplate more than one embodiment. The following detailed description is, therefore, not to be taken in a limiting sense, and the scope is defined only by the appended claims, along with the full scope of legal equivalents to which such claims are entitled.

The present subject matter delivers cardiac protective pacing therapy (CPPT) to protect the heart from injuries and/or to treat existing injuries. CPPT may also be referred to as an intermittent pacing therapy (IPT). The pacing system receives a set of inputs and delivers optimized cardiac protection pacing tailored for each of different purposes. The system automatically adjusts heart rate to optimize cardiac protection pacing in a closed-loop system. The system delivers electrical stimulation to modulate myocardial strain for anti-atherosclerosis therapy, to provide therapy for MI, to provide therapy for angina, and/or to provide therapy for co-morbidities related to neural imbalance.

Autonomic tone may be modulated by exciting or inhibiting an autonomic neural target. Embodiments of the present subject mater modulate autonomic tone using CPPT. Physiology associated with CPPT is discussed below.

The sinoatrial (SA) node generates electrical impulses that propagate through an electrical conduction system to various regions of the heart to excite the myocardial tissues of these regions. An intrinsic heart rhythm may be a normal rhythm or an abnormal rhythm. Coordinated delays in the propagations of the electrical impulses in a normal electrical conduction system cause the various portions of the heart to contract in synchrony. Synchrony, as used herein, indicates a coordinated contraction of the various portions of the heart to result in efficient pumping functions. Synchrony does not indicate that all of the portions of the heart contract at the same time.

Abnormal electrical conduction and/or deteriorated myocardial tissue cause asynchrony (no coordinated timing) between the various portions of the heart, which result in inefficient pumping functions. The present subject matter uses cardiac protective pacing therapy (CPPT) to provide a cardiac conditioning therapy to improve autonomic balance, and thus improve the health of the heart. CPPT is an intermittent pacing therapy that paces the heart in such a manner as to intentionally augment a heart's existing stress and/or redistribute the stress during intermittent periods. When the heart is stressed with CPPT, the heart is forced to work harder for brief periods in comparison to a time when CPPT is not applied to the heart. The paced heart works harder in local regions of the heart away from a site where the stress-inducing pacing pulses are delivered. For example, a stressed heart may be paced to beat faster and/or more asynchronous (less coordinated). By way of example and not limitation, various CPPT embodiments increase the pacing rate for the right atrium, increase the pacing rate for the right ventricle, shorten an AV delay, and/or lengthen the VV delay, thereby augmenting cardiac stress (or stress on the myocardium). Increasing the intensity of the CPPT may involve further increasing the pacing rate of the right atrium or right ventricle, further shortening the AV delay to be more different from the intrinsic rate without CPPT, altering the pacing site, and/or further lengthening of the VV delay to be more different from the intrinsic rate without CPPT. In patients who have dysynchrony and receive biventricular pacing for the dysynchrony, cardiac stress can be increased by discontinuing the biventricular pacing during the sequence of stress inducing pacing pulses. Decreasing the intensity of the CPPT may involve altering the pacing site, may involve reducing the pacing rate of the right atrium or right ventricle closer to the intrinsic rate, may involve increasing the AV delay closer to the intrinsic AV delay, and/or may involve shortening the VV delay closer to the intrinsic VV delay (whether or not the intrinsic rhythm is normal or abnormal). Delivering CPPT with higher intensity corresponds to increasing the sympathetic response during the CPPT.

### Diseases

The present subject matter can be used to prophylactically or therapeutically treat various diseases by modulating autonomic tone and/or inducing myocardial strain. Examples of such diseases or conditions include hypertension, cardiac remodeling, heart failure, atherosclerosis, MI, angina, and co-morbidities related to neural imbalance, such as sleep apnea.

Hypertension is a cause of heart disease and other related cardiac co-morbidities. Hypertension occurs when blood vessels constrict and/or become non-compliant or when cardiac output or blood volumes increase. As a result, the heart works harder to maintain flow at a higher blood pressure, which can contribute to myocardial remodeling and heart failure. Hypertension generally relates to high blood pressure, such as a transitory or sustained elevation of systemic arterial blood pressure to a level that is likely to induce cardiovascular damage or other adverse consequences. Hypertension has been defined as a systolic blood pressure above 140 mm Hg or a diastolic blood pressure above 90 mm Hg. Consequences of uncontrolled hypertension include, but are not limited to, retinal vascular disease and stroke, left ventricular hypertrophy and failure, MI, dissecting aneurysm, and renovascular disease. A large segment of the general population, as well as a large segment of patients implanted with pacemakers or defibrillators, suffer from hypertension. The long term mortality as well as the quality of life can be improved for this population if blood pressure and hypertension can be reduced. Many patients who suffer from hypertension do not respond to treatment, such as treatments related to lifestyle changes and hypertension drugs.

Following MI or other cause of decreased cardiac output, a complex remodeling process of the ventricles occurs that involves structural, biochemical, neurohormonal, and electrophysiologic factors. Ventricular remodeling is triggered by a physiological compensatory mechanism that acts to increase cardiac output due to so-called backward failure which increases the diastolic filling pressure of the ventricles and thereby increases the so-called preload (i.e., the degree to which the ventricles are stretched by the volume of blood in the ventricles at the end of diastole). An increase in preload causes an increase in stroke volume during systole, a phenomena known as the Frank-Starling principle. When the ventricles are stretched constantly due to the increased preload over a period of time, however, the ventricles become dilated. The enlargement of the incessant ventricular volume causes increased ventricular wall stress at a given systolic pressure. Along with the increased pressure-volume work done by the ventricle, this acts as a stimulus for hypertrophy of the ventricular myocardium. The disadvantage of dilatation is the extra workload imposed on normal, residual myocardium and the increase in wall tension (Laplace's Law) which represent the stimulus for hypertrophy. If hypertrophy is not adequate to match increased tension, a vicious cycle ensues which causes further and progressive dilatation. As the heart begins to dilate, afferent baroreceptor and cardiopulmonary receptor signals are sent to the vasomotor central nervous system control center, which responds with hormonal secretion and sympathetic discharge. It is the combination of hemodynamic, sympathetic nervous system and hormonal alterations (such as presence or absence of angiotensin converting enzyme (ACE) activity) that ultimately account for the deleterious alterations in cell structure involved in ventricular remodeling. The sustained stresses causing hypertrophy induce apoptosis (i.e., programmed cell death) of cardiac muscle cells and eventual wall thinning which causes further deterioration in cardiac function. Thus, although ventricular dilation and hypertrophy may at first be compensatory and increase cardiac output, the constant myocardial stress ultimately results in both systolic and diastolic dysfunction (decompensation). It has been shown that the extent of ventricular remodeling is positively correlated with increased mortality in post-MI and heart failure patients.

Atherosclerosis begins with the appearance of cholesterol-laden macrophages (foam cells) in the intima of an artery. Smooth muscle cells respond to the presence of lipid by proliferating, under the influence of platelet factors. A plaque forms at the site, consisting of smooth muscle cells, leukocytes, and further deposition of lipid; in time the plaque becomes fibrotic and may calcify. Expansion of an atherosclerotic plaque leads to gradually increasing obstruction of the artery and ischemia of tissues supplied by it. Ulceration, thrombosis, or embolization of a plaque, or intimal hemorrhage and dissection, can cause more acute and severe impairment of blood flow, with the risk of infarction. Treatment of atherosclerosis includes balloon stretching, laser ablation, or surgical removal of plaques, and various bypass and grafting procedures. Current preventive measures for atherosclerosis include regular vigorous exercise, a diet low in fat and cholesterol, maintenance of a healthful weight, avoidance of tobacco, and use of pharmacologic agents as indicated.

Besides causing an MI, coronary artery disease (CAD) can also produce lesser degrees of cardiac ischemia due to the narrowing of a coronary artery lumen by atherosclerotic plaque. When blood flow and oxygen supply to the heart is reduced, patients often experience chest pain or discomfort, referred to as angina pectoris. Angina pectoris serves as a useful warning of insufficient myocardial perfusion which can lead to the more serious situation such as a heart attack or cardiac arrhythmia. Patients who experience anginal episodes are commonly treated either with medication or by surgical revascularization.

Examples of targeted co-morbidities related to neural imbalance include, but are not limited to, central respiratory diseases (sleep disordered breathing, apnea), atrial fibrillation (AF) burden, hypertension, and renal dysfunction. Central respiratory diseases include disorders that affect breathing during sleep or while a person is awake. Central respiratory diseases are associated with incorrect sensing of carbon dioxide or oxygen levels in the blood. If nerve receptors do not send the correct neural signals, in essence deceiving the brain by reporting incorrect levels of carbon dioxide or oxygen, an incidence of a central respiratory disease can occur. The brain responds by slowing breathing, and even ceasing breathing in extreme cases. Respiratory disorders during sleep and during the day include central sleep apnea or hypopnea and periodic breathing or dyspnea, respectively. Central sleep apnea refers to the cessation of breathing during sleep, and hypopnea refers to abnormally slow or shallow breathing during sleep. Both conditions have serious health consequences, including association with cardiac arrhythmias.

Heart failure refers to a clinical syndrome in which cardiac function causes a below normal cardiac output that can fall below a level adequate to meet the metabolic demand of peripheral tissues. Heart failure may present itself as congestive heart failure (CHF) due to the accompanying venous and pulmonary congestion. Heart failure can be due to a variety of etiologies such as ischemic heart disease. Heart failure patients have reduced autonomic balance, which is associated with LV dysfunction and increased mortality. Modulation of the sympathetic and parasympathetic nervous systems has potential clinical benefit in preventing remodeling and death in heart failure and post-MI patients. Direct electrical stimulation can activate the baroreflex, inducing a reduction of sympathetic nerve activity and reducing blood pressure by decreasing vascular resistance. Sympathetic inhibition and parasympathetic activation have been associated with reduced arrhythmia vulnerability following MI, presumably by increasing collateral perfusion of the acutely ischemic myocardium and decreasing myocardial damage.

According to an embodiment, the present subject matter modulates autonomic tone using CPPT. Preconditioning of the myocardium occurs as a prophylactic therapy in preparation for an anticipated event. For example, the myocardium can be preconditioned in anticipation for surgery, or can be preconditioned based on observed or detected events that indicate an increased probability of an upcoming ischemic event. Examples of such events include a previous MI and angina. Prophylactic conditioning can be used to modulate autonomic tone from higher sympathetic tendencies toward an autonomic balance to improve the health of a patient prone to heart failure, hypertension and remodeling. Postconditioning of the myocardium occurs as a therapeutic treatment to a disease. For example, postconditioning of the myocardium can limit the expansion of the size of an infarct area caused by the ischemic event. For example, the postconditioning therapy can be triggered based on commands received from a patient or physician after observing an MI, or a physician can deliver postconditioning therapy after a surgical procedure for which the heart was stopped. In an embodiment, the device detects an ischemic event or other event indicated for postconditioning therapy, and automatically delivers the postconditioning therapy. The postconditioning therapy can occur during the time of reperfusion, for a time after reperfusion, or during and for a time after reperfusion. IPT and post-conditioning are both CPPT using the same or at least same-type pacing algorithm, with the difference being time of delivery. IPT refers to a chronic (long-term) delivery of the CPPT. Post-conditioning refers to an acute (short-term) delivery of the CPPT typically applied right after an ischemic event such as an acute MI.

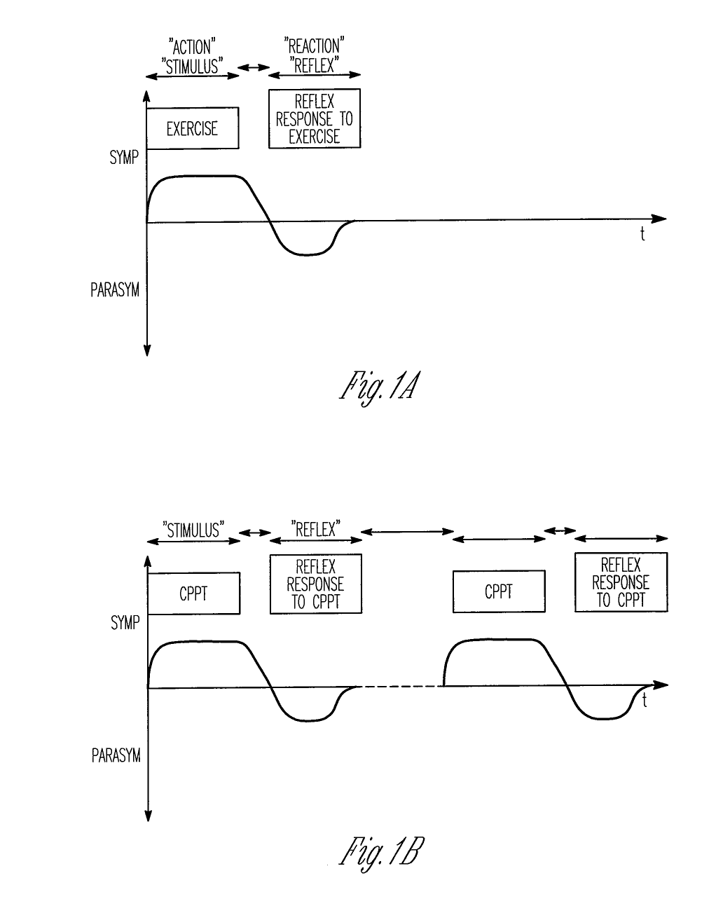

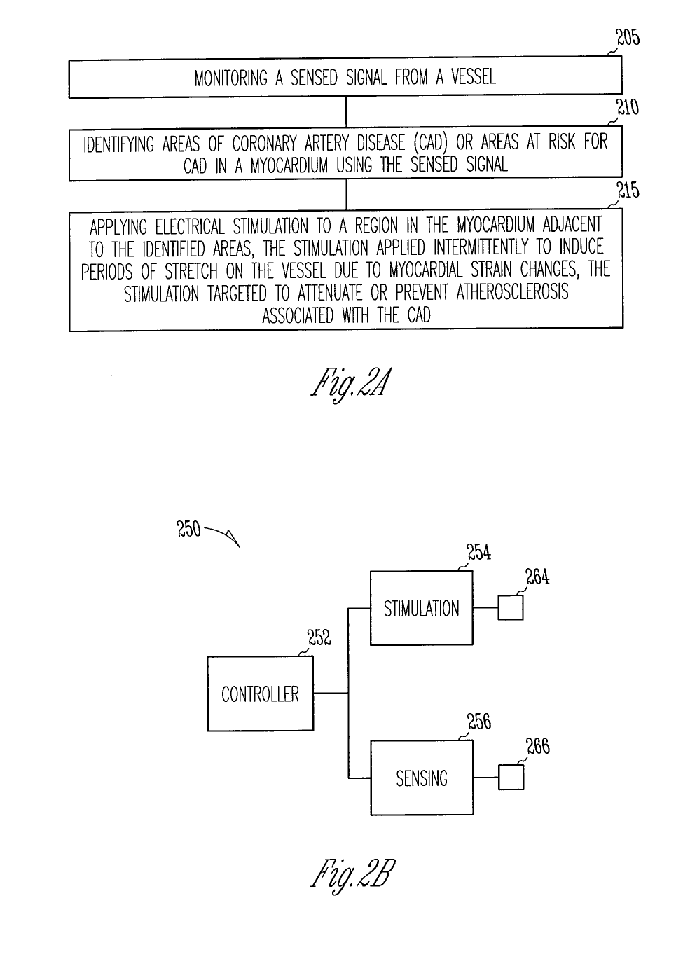

A cardiac conditioning therapy may also be referred to as a CPPT, as it is protects against the deleterious effects of an autonomic tone with an undesirably high sympathetic tendency. The cardiac conditioning therapy may mimic the effects of exercise. FIG. 1A illustrates the autonomic response to a period of exercise. Exercise is a stimulus that increases the sympathetic response. After the period of exercise ends, a reflex response to the exercise increases the parasympathetic tone. The parasympathetic response appears to be a reaction to the sympathetic action of exercise. Those of ordinary skill in the art will understand that the illustrated graph is a simple illustration. The horizontal axis represents time, and the vertical axis represents the autonomic tone. For simplicity, the value of the vertical axis corresponding to the horizontal axis represents the autonomic balance (the balance between the sympathetic and parasympathetic neural activity). Those of ordinary skill in the art will know that, over time, as the health of the heart improves and the autonomic balance improves by having a more parasympathetic tone, the horizontal axis (representing the autonomic balance) will trend more toward the parasympathetic tone. By way of an everyday example of exercise, it is noted that a runner's resting heart rate tends to lower as the runner's conditioning improves. This example indicates that running, which temporarily increases sympathetic tone as evidenced by an increased heart rate, will trend the autonomic balance of the runner toward a more parasympathetic tone.

FIG. 1B illustrates the autonomic response to a period of CPPT. Similar to the period of exercise, CPPT is a stimulus that increases the sympathetic response during the period of pacing, and results in a reflex response that increases parasympathetic tone after the pacing ends. As illustrated, the CPPT functions as a stimulus that provides a sympathetic component (action) that generates a desired parasympathetic reflex (reaction to the action). A cardiac conditioning therapy may correspond to recommended exercises periods (e.g. 30 to 60 minutes, two times per day). Various therapy durations and frequencies can be used. Various cardiac conditioning therapies are programmed according to a schedule. Various cardiac conditioning therapies are programmed to occur after a detected event.

### Anti-Atherosclerosis Therapy

Pacing can increase fiber length (stretch) in targeted regions. Intermittent pacing therapy (IPT) has been demonstrated to attenuate remodeling (LV volume, mass) and improve functional capacity. IPT can be provided in a variety of modes, including VVI, VOO, DOO, short AV delay pacing, or long VV delays. IPT modes for dual-chamber CRM devices include AV delay modulation to create intermittent stress, as described above. Pacing can modulate the mechanical strain characteristics of the myocardium. These strain forces can also be transferred to adjacent coronary arteries because they are tethered to the myocardium. Intermittently inducing changes in strain characteristics of coronary arteries disrupts or attenuates the formation of plaque in the arteries. Pacing applied intermittently to induce short periods of stretch on coronary arteries due to stimulating the adjacent myocardium is therapeutic in preventing or attenuating atherosclerosis. Coronary arteries tethered to adjacent myocardium are affected by pacing induced myocardial strain changes. According to various embodiments, applying IPT results in intermittent longitudinal stretch of the artery, disrupting plaque attachment and/or infiltration of endothelium. Vessel stretch also causes the release of nitric oxide (NO) which promotes circumferential vasodilation.

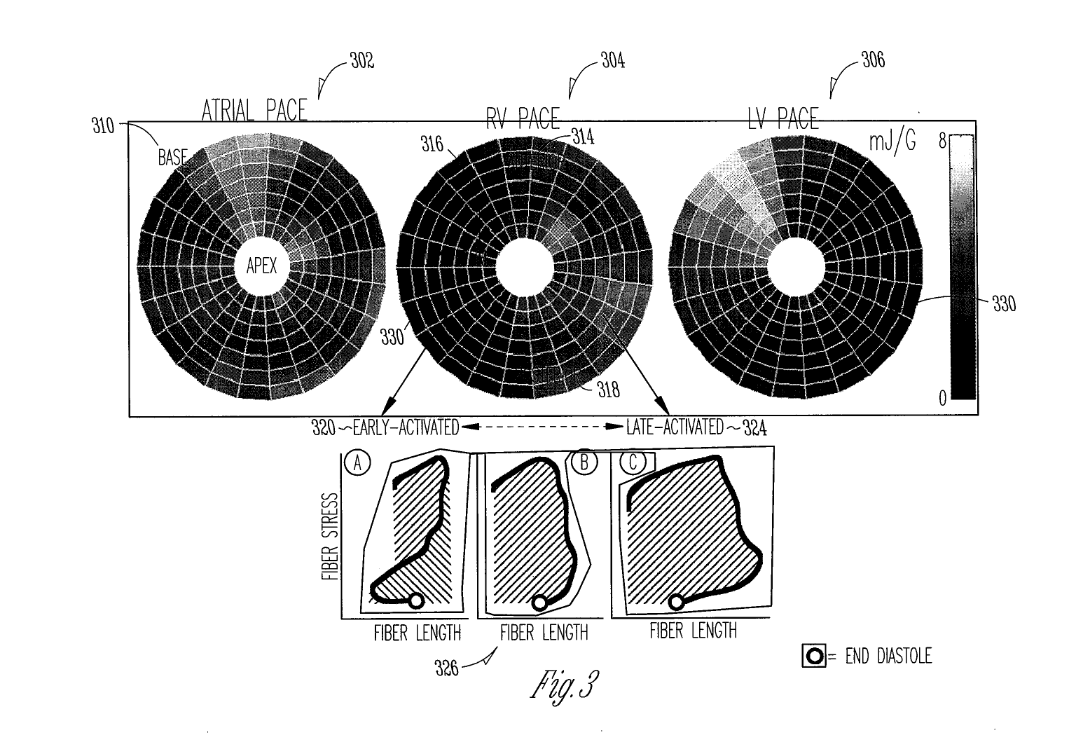

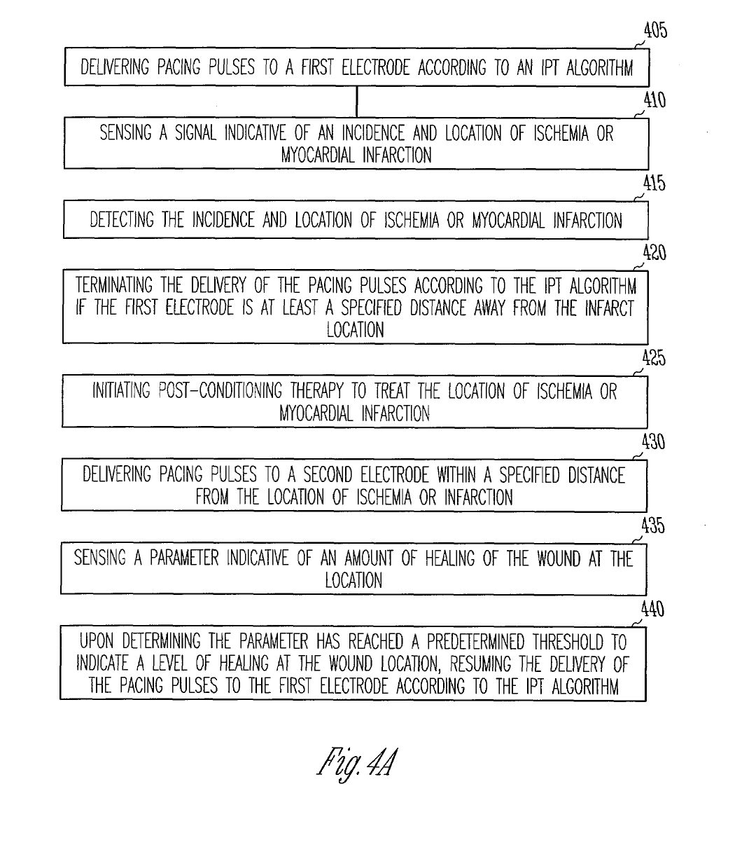

FIG. 2A is a flow chart illustrating an embodiment of a method for delivering pacing pulses for treating atherosclerosis. At 205, a sensed signal from a vessel is monitored, and areas of coronary artery disease (CAD) or areas at risk for CAD in a myocardium are identified using the sensed signal, at 210. Examples of vessels monitored include left and right coronary arteries. According to an embodiment, the sensed signal includes a signal indicative of blood flow in the vessel. CAD can be detected by monitoring blood follow in the arteries, and detecting a flow rate at a programmable threshold below the normal rate. At 215, electrical stimulation is applied to a region in the myocardium adjacent to the identified areas. According to an embodiment, the electrical signal can be delivered remotely, as dyssynchrony increases stretch and can be accomplished from remote locations. The stimulation is applied intermittently to induce periods of stretch on the vessel due to myocardial strain changes. According to various embodiments, stimulation parameters are selected to alter the timing of the pacing pulse to deliver the desired stress and/or stretch. The stimulation is targeted to attenuate or prevent atherosclerosis associated with the CAD. Pacing leads and electrodes are positioned remote from vessels with CAD to increase strain (see FIG. 3), in various embodiments.

According to various embodiments, the applied electrical stimulation includes ventricular pacing, bi-ventricular pacing, and/or intermittent ventricular pacing. Ventricular pacing can be applied at a heart rate (HR) greater than intrinsic HR, in various embodiments. In one embodiment, ventricular pacing is applied to produce a heart rate (HR) at least about 10 beats per minute greater than intrinsic HR. In another embodiment, ventricular pacing is applied to produce a heart rate (HR) at least about 12 beats per minute greater than intrinsic HR. Intermittent pacing includes multiple short periods of on/off pacing cycles to increase strain changes, in various embodiments. Applying electrical stimulation includes alternating between pacing sites to increase strain changes, in an embodiment.

FIG. 2B is a block diagram illustrating an embodiment of a medical device for delivering pacing pulses for treating atherosclerosis. The medical device 250 for use in a body includes a sensing circuit 256 to receive sensed signals to identify areas of coronary artery disease (CAD) or areas at risk for CAD using the sensed signals. The device also includes a pacemaker circuit 254 adapted to deliver an electrical signal through at least one electrode 264 to a myocardial target adjacent to the identified areas. According to various embodiments, a controller 252 communicates with the sensing circuit 256 and controls the pacemaker circuit 254 to provide intermittent electrical stimulation to the myocardial target to induce periods of stretch on the vessel due to myocardial strain changes. The stimulation is targeted to attenuate or prevent atherosclerosis associated with the CAD. One or more sensors 266 are electrically connected to the sensing circuit 256. In an embodiment, the electrodes 264 are electrically connected to the sensing circuit 256, and are further adapted to function as sensors. FIGS. 10-13 provide other examples of apparatus for delivering pacing for treating atherosclerosis.

According to various embodiments, the medical device includes an implantable cardiac rhythm management (CRM) pulse generator (PG) with intra-cardiac leads. The medical device can also include a single chamber pacemaker adapted to treat localized CAD. A single chamber device can be used for patients with no other bradycardia or tachycardia indications. In on embodiment, a single chamber device is paced (VVI) at a programmably higher HR than intrinsic, for example 10-12 beats per minute. Multiple short periods of on/off cycles maximize strain changes. In other embodiments, the medical device includes a biventricular pacemaker adapted to treat multiple-region CAD. In one embodiment, pacing is alternated between multiple sites to maximize strain. Other bradycardia or tachycardia devices can also be used for patients with appropriate indications. According to various embodiments, the sensed signals include signals indicative of blood flow in the vessel. Other sensed signals can be used to identify patients and substrates which will respond to intermittent pacing. For example, the sensed signals can include signals from a nuclear exam, signals from an echocardiogram, and/or signals from an intravascular ultrasound (IVUS) exam in various embodiments. A non-invasive ultrasound exam of brachial artery flow-mediated dilation correlates with CAD. In a closed-loop system, therapy titration is enhanced by sensing inputs for inflammatory markers, plasma markers of endothelial function and/or ischemia detection, and adjusting therapy based on the sensed parameter. In an embodiment, stimulation can be provided to stretch directly on the vessel. In another embodiment, stimulation is provided to result in an indirect activation of cardioprotective mechanisms, such as the anti-inflammatory effect of IPT.

FIG. 3 illustrates the effect of pacing on myocardial strain patterns. Atrial pacing 302, right ventricle (RV) pacing 304 and left ventricle (LV) pacing 306 are depicted. The base 310 and apex 312 of strain patterns are shown, as well as the anterior 314, septum 316 and posterior 318 myocardial locations. Applying electrical stimulation to pace from target locations 330 shows strain in fibers on a continuum from early activated areas 320 through the late activated areas 324 and areas in between 326. As shown, pacing can increase fiber length (stretch) in targeted regions.

### Infarction Therapy

Pacing therapies to thicken an injured region, such as an infarct region, will decrease wall tension and reduce long-term remodeling. After an MI, as an infarct region heals, the present subject matter moves a pacing site from the infarct/border zone to a remote zone for IPT. Pacing a remote zone causes the infarct/border zone to be under short periods of increased stress and promote border zone thickening (wound healing) and reduce wall tension. Wound healing will be aided by the increased stress, causing hypertrophy and increase fibrosis. The present subject matter uses an implantable CRM device (such as a CRT device or pulse generator (PG)) that delivers intermittent stimulation to prevent remodeling following an MI. Ischemia or MI is detected using electrocardiogram derivatives, lead impedance, or other thresholds. Upon detection, IPT delivered to a pacing site in the infract region is discontinued and post-conditioning is initiated and delivered to a pacing site remote from the infarct region by a specified distance. Wound healing is estimated, and upon sufficient wound healing (after 1-2 weeks), pacing is resumed from a remote zone (either intermittently or continuously), and can be combined with other types of pacing like OPIS (pacing developed by the Ohio pacing infarct study) or MENDMI (discussed below).

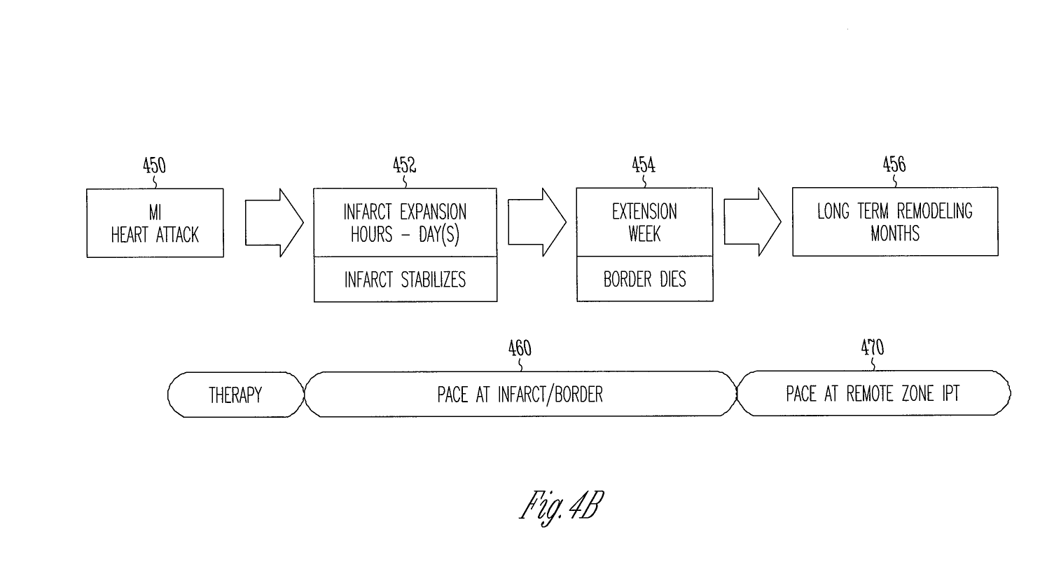

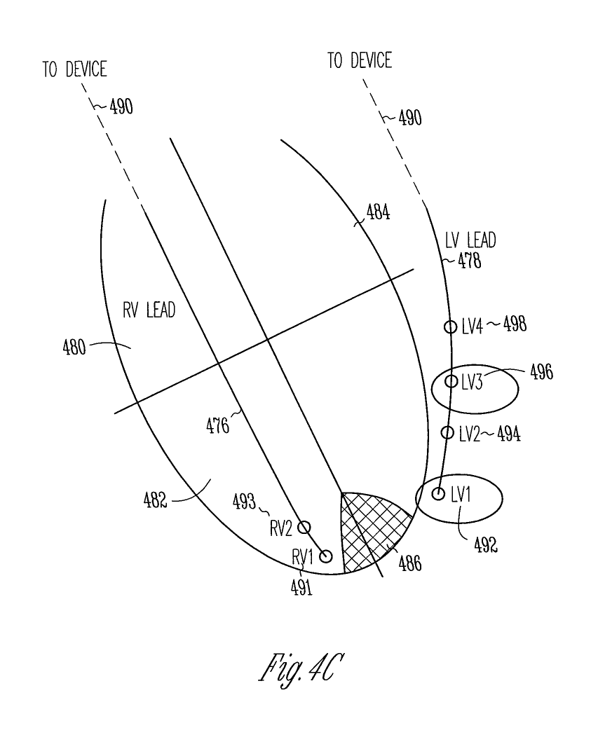

FIG. 4A is a flow chart illustrating a method for ventricular pacing to treat MI, according to various embodiments of the present subject matter. At 405, pacing pulses are delivered to a first electrode according to an IPT algorithm. At 410, a signal is sensed indicative of an incidence and location of ischemia or myocardial infarction, and the incidence and location of ischemia or myocardial infarction is detected, at 415. At 420, the delivery of the pacing pulses according to the IPT algorithm is terminated if the first electrode is at least a specified distance away from the infarct location to prevent rupture. Post-conditioning therapy is initiated, at 425, to treat the location of ischemia or myocardial infarction of ischemia or infarction. At 430, pacing pulses are delivered to a second electrode within a specified distance from the location of ischemia or myocardial infarction. A parameter is sensed indicative of an amount of healing of the wound at the location of ischemia or myocardial infarction, at 435. Upon determining the parameter has reached a predetermined threshold to indicate a level of healing at the wound location, the delivery of the pacing pulses to the first electrode is resumed according to the IPT algorithm, at 440.

According to various embodiments, sensing a signal includes sensing lead impedance. Sensing a parameter indicative of the amount of healing includes using pacing thresholds, a mechanical sensor, and/or a hemodynamic sensor, in various embodiments. Initiating post-conditioning therapy includes ventricular pacing at the location of ischemia or MI, in an embodiment.

FIG. 4B illustrates a method for pacing to treat MI, according to various embodiments of the present subject matter. A timeline is depicted illustrating treatment of a detected MI. An MI occurs (450) and subsequently (452) the infarct expands over the course of hours and days after the MI, eventually stabilizing and then over the course of the weeks after the MI (454) the border disappears. Long term remodeling occurs (456) over the course of months after the MI. During the initial time periods (452 and 454) after the MI, pacing (such as post-conditioning pacing) is provided at the infarct region or border of the region, at 460. Eventually, as the infarct heals (time period 456), IPT is provided (at 470) remote from the infarct region to cause the infarct/border zone to be under increased workload during IPT and over time will induce thickening of the border zone. Stress on the non-contracting surrounding myocardium releases neuro-hormones and cytokines that stimulate replacement fibrosis and aid in wound healing.

FIG. 4C illustrates electrode location for pacing to treat MI, according to an embodiment. A heart 480 is depicted having right ventricle (RV) 482 and left ventricle (LV) 484, and having an infarct region 486. The depicted therapy system includes a biventricular pacing cardiac rhythm therapy (CRT) device (not shown) equipped with an MI detector and IPT therapy delivery capability, and having an RV lead 476 and an LV lead 478. Both leads are connected to the device at their proximal ends 490. At the distal ends of the leads, multiple electrodes are electrically connected, and positioned along the leads at varying distances from the infarct region 486. The RV lead 476 has an RV1 electrode 491 near the infarct region and an RV2 electrode 493. The LV lead 478 includes an LV1 electrode 492 near the infarct region, an LV2 electrode 494, and LV3 electrode 496 and an LV4 electrode 498 furthest from the infarct region. According to an embodiment, an MI is detected and existing IPT, if any, is discontinued. Post-conditioning or post-MI therapy is initiated near the infarct region 486, in this case using pacing electrode LV1 492. Therapy such as MENDMI (electrical stimulation to prevent myocardial enlargement and dilation post-MI) is delivered using electrode LV1 492. Healing of the infarct region is subsequently detected, such as by monitoring changes in pacing thresholds. Upon detection of a predetermined level of MI healing, IPT is resumed from a remote site, such as LV3 496. Continuous pacing at LV1 492 is done in combination with intermittent pacing at LV3 496, in various embodiments.

According to various embodiments, wound healing can be estimated sensing electrical characteristics, mechanical characteristics (such as sonogram, XL, stress, mass), hemodynamic sensors (pressure, CO, contractility), or could be externally estimated by a physician or patient. Upon sensing a parameter threshold indicative of wound healing, IPT can be delivered by pacing a remote area (from infarct region) intermittently or continuously. Pacing can be in VDD or DDD mode with a relatively high rate. In an embodiment, pacing at short AV delay can be delivered to increase stress in the remote area. Pacing at a high output (voltage, amplitude, and/or width) an also be delivered.

In one embodiment, a medical device for treating MI is provided. The medical device, such as the device in FIG. 2B combined with the lead structure of FIG. 4C, includes a sensing circuit adapted to receive sensed signals indicative of an incidence and location of ischemia or myocardial infarction and to detect the incidence and location of ischemia or myocardial infarction, and further adapted to sense a parameter indicative of an amount of healing at the location of ischemia or myocardial infarction. The device also includes cardiac pacing lead, including a first electrode placed a specified distance away from the infarct location, and a second electrode placed nearer the location of ischemia or myocardial infarction than the first electrode. The device further includes a pacemaker circuit connected to the pacing lead and adapted to deliver an electrical signal at a programmed level for a programmed duration. A controller communicates with the sensing circuit and controls the pacemaker circuit to provide electrical stimulation through the first electrode to deliver IPT, through the second electrode to deliver post-conditioning therapy subsequent to sensing an incidence of ischemia or myocardial infarction, and, subsequent to sensing a programmable threshold amount of healing, through the first electrode to resume delivery IPT.

### Angina Therapy

A patient with angina is limited in their activity due to the accompanying symptoms. Over time, angina can lead to angiogenesis. The present subject matter proposes to provide pacing (either continuous or intermittent) from a region remote from an angina region to promote mass redistribution and angiogenesis at the angina region. Pacing a remote region will increase work load/stress/strain at the angina region. In various embodiments, a two chamber device can be used to deliver continuous pacing at the site of angina to unload, and to deliver intermittent pacing (such as CPPT) from the remote site to provide stress to the angina region. The remote site is chosen such that pacing would create a region of increased stress at the angina site. Different CPPT modes can be used to promote angiogenesis and for maintenance. The device can monitor for activity level to stop CPPT or switch CPPT modes, in various embodiments. Devices suitable for delivering the therapy discussed include the device of FIG. 10, and CRT or PG (in combination with a nerve stimulator) devices.

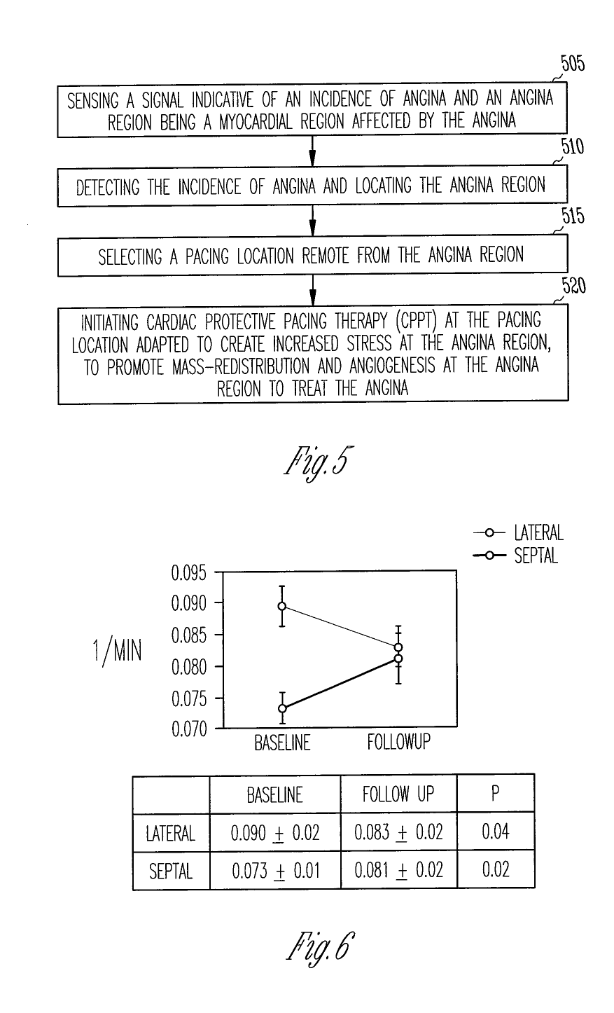

FIG. 5 is a flow chart illustrating a method for treating angina, according to an embodiment of the present subject matter. At 505, a signal is sensed indicative of an incidence of angina and an angina region being a myocardial region affected by the angina. The incidence of angina is detected and the angina region is located, at 510. At 515, a pacing location is selected remote from the angina region. Cardiac protective pacing therapy (CPPT) is initiated at the pacing location, at 520. The CPPT is adapted to create increased stress at the angina region, to promote mass-redistribution and angiogenesis at the angina region to treat the angina.

According to various embodiments, sensing a signal to detect an incidence of angina can be accomplished using a device-based method and/or an imaging-based method. A device-based method can include using electrograms (EGMs) in bipolar mode to identify angina. A physician can input the information regarding the location of the angina, in an embodiment. CPPT can include continuous or intermittent pacing therapy. CPPT can be delivered at specific times of the day, and may be patient initiated, in an embodiment. CPPT can be delivered using regular on/off pacing cycles, such as three cycles of on/off pacing over 60 minutes in an embodiment. In one embodiment, initiating CPPT includes using ST elevation to cause mild ischemia, and to change the pacing mode and/or duration of pacing. According to various embodiments, the method further comprises monitoring patient activity level and using the monitored level as an index of therapy efficacy in a closed loop system. Short-term monitoring for the closed loop system can include monitoring EGMs and/or monitoring for arrhythmia, in various embodiments. CPPT can be delivered by pacing remotely from the angina region intermittently, pacing in VDD or DDD mode with a high rate, pacing at short AV delay to increase remote stress, or pacing at high output (voltage, amplitude and/or width).

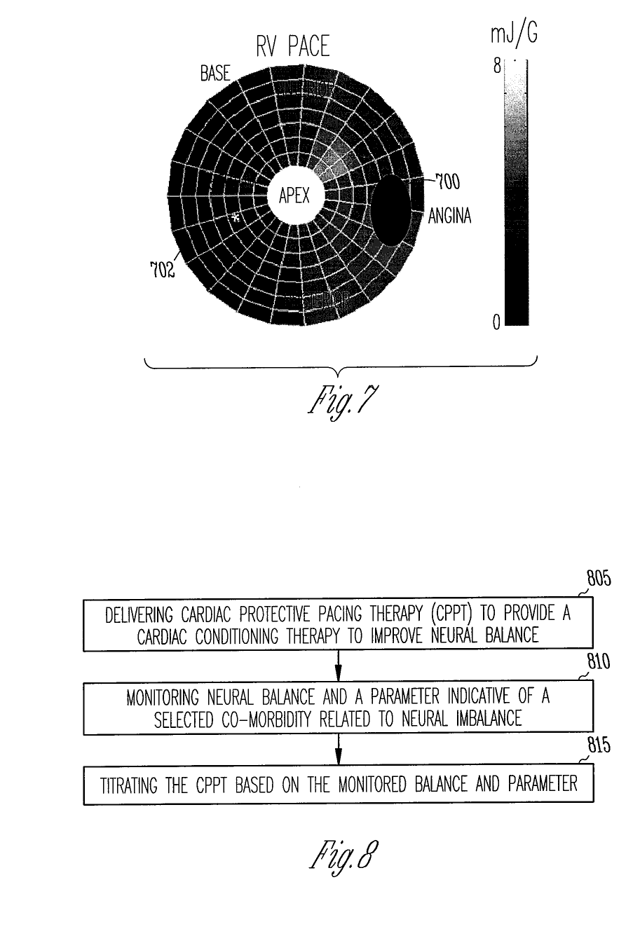

FIG. 6 illustrates the effect of cardiac resynchronization therapy (CRT) on regional myocardial oxygen consumption. CRT has been shown to renormalize region myocardial oxygen consumption in patients with left bundle branch block (LBBB). The depicted graph shows the inverse relationship of regions C-acetate clearance in the lateral and septal wall before and after resynchronization therapy. The reverse remodeling of regional myocardial oxygen consumption is statistically significant. Thus, a region with more strain develops more collateral flow. FIG. 7 illustrates the effect of CPPT pacing on strain patterns relative to an angina region, according to various embodiments. The depicted embodiment paces from a pacing site 702 remote from an angina region 700. CPPT is delivered at a remote region and at a slightly elevated rate to cause increase work/strain at the angina region and promote angiogenesis.

In one embodiment, a medical device for treating angina is provided. The device, such as the device in FIG. 2B, includes a sensing circuit to receive sensed signal indicative of an incidence of angina and an angina region being a myocardial region affected by the angina. The device also includes a pacemaker circuit adapted to deliver an electrical signal through at least one electrode to a pacing location remote from the angina region. According to various embodiments, the device further includes a controller to communicate with the sensing circuit and to control the pacemaker circuit to provide CPPT to the pacing location adapted to create increased stress at the angina region, to promote mass-redistribution and angiogenesis at the angina region to treat the angina.

### Disease Prevention Therapy

CPPT impacts the short and long-term neurological balance. Transient atrial overdrive pacing has been shown to improve sleep apnea-hypopnea without disturbing sleep structure. Sympathetic stimulation can occur during the CPPT pacing followed by a parasympathetic surge. This shift in neurological balance can help prevent or reduce the symptoms of co-morbidities that are related to the neurological imbalance. Examples of targeted co-morbidities include, but are not limited to, sleep disordered breathing, apnea, atrial fibrillation (AF) burden, hypertension, and renal dysfunction. Depending on the co-morbidity of interest, the physician and/or the device can select a parameter to monitor.

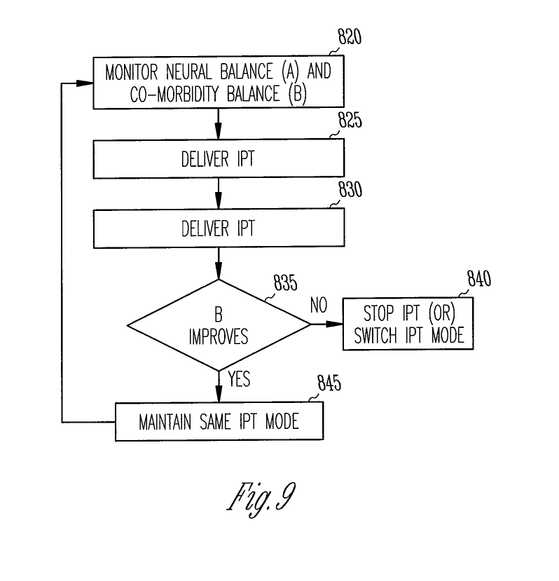

FIG. 8 is a flow chart illustrating an embodiment of a method for treating co-morbidities related to neural imbalance. At 805, cardiac protective pacing therapy (CPPT) is delivered to provide a cardiac conditioning therapy to improve neural balance. At 810, neural balance and a parameter indicative of a selected co-morbidity related to neural imbalance are monitored, and the CPPT is titrated based on the monitored balance and parameter at 815.

According to various embodiments, monitoring a parameter of interest includes allowing a physician to select the co-morbidity. Various co-morbidities can be monitored, including sleep disordered breathing, apnea, atrial fibrillation (AF) burden, hypertension, and renal dysfunction. Parameters that can be monitored include apnea-hypopnea index (AHI) for apnea, pulmonary arterial pressure (PAP), V tachy burden for VT storms or PVCs, and number of episodes and/or duration for AF burden, for example. According to various embodiments, titrating the CPPT includes continuing CPPT unchanged if the sensed parameter improves, discontinuing CPPT unchanged if the sensed parameter degrades or is unchanged, and continuing CPPT with a new therapy mode if the sensed parameter degrades or is unchanged. For example, in one embodiment apnea is the co-morbidity of interest and AHI is monitored. CPPT mode, for example DDD pacing at 80 beats per minute (bpm), is turned on for a short time period and AHI is monitored during the off period. If the AHI improves during the off period, CPPT is continued in the current mode. If the AHI does not change during the off period, IPT mode is changed to increase stress, for example DDD pacing at 90 bpm. If the AHI increases (gets worse) during the off period, IPT mode is switched off, or in the alternative CPPT mode is switched to decrease stress, for example DDD pacing at 70 bpm.

Devices suitable for delivering the therapy discussed include the device of FIG. 10, and CRT or PG (in combination with a nerve stimulator) devices. According to various embodiments, the device includes ventricular and/or atrial leads, and is capable of detecting status of co-morbidities and symptoms, delivering CPPT (programmable) and titrating CPPT based on co-morbidities and symptoms. The titration can be automatic by the device controller or remote by the physician from a programmer or monitoring type external device. A single chamber device can deliver CPPT with different rates of overdrive pacing. A dual chamber device can also deliver CPPT with shortened AV delay or varied VV delay, or a combination of overdrive pacing and shorted AV delay.

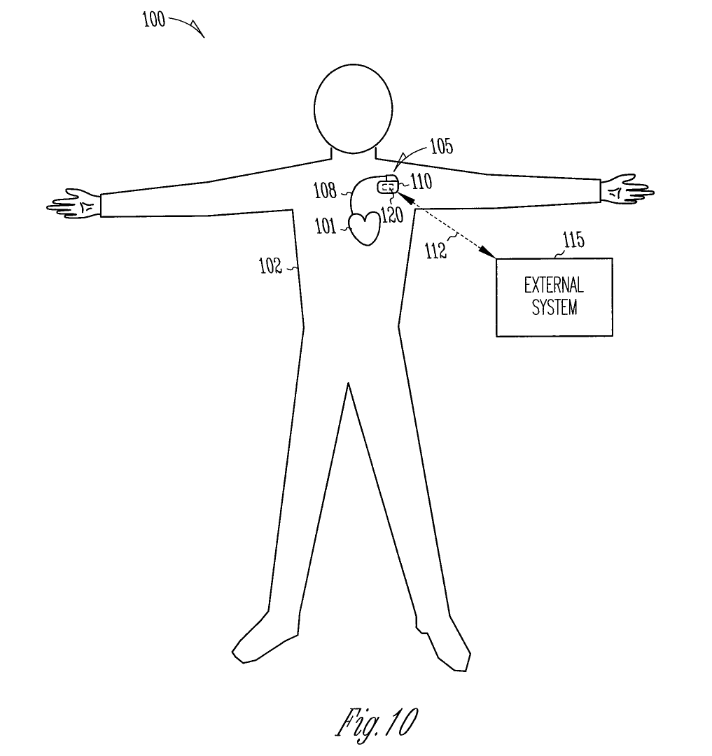

FIG. 9 is a flow chart illustrating an embodiment of a method using intermittent pacing to treat co-morbidities related to neural imbalance in a closed loop system. At 920, neural balance (A) and a parameter (B) of interest for a selected co-morbidity related to neural imbalance are monitored. At 925, a session of cardioprotective IPT is delivered. Changes in parameters related to neural balance and the selected co-morbidity are monitored at 930. If the parameter related to the co-morbidity improves at 935, the same IPT mode is used at 945 and the parameters are again monitored at 920. If the parameter related to the co-morbidity does not improve at 935, the IPT is stopped or continued in a different mode at 940.

In one embodiment, a medical device for treating co-morbidities related to neural imbalance is provided. The device, such as the device in FIG. 2B, includes a sensing circuit adapted to receive sensed signals indicative of neural balance and adapted to receive sensed parameters indicative of a selected co-morbidity related to neural imbalance. The device also includes a pacemaker circuit adapted to deliver an electrical signal through at least one electrode to a target location. The device further includes a controller adapted to communicate with the sensing circuit and to control the pacemaker circuit to provide CPPT to the target location, and further adapted to titrate the CPPT based on the sensed signals and parameters.

### Pacing Apparatus

FIG. 10 is an illustration of an embodiment of a cardiac rhythm management (CRM) system 100 and portions of an environment in which system 100 is used. System 100 includes an implantable system 105, an external system 115, and a telemetry link 112 providing for communication between implantable system 105 and external system 115.

Implantable system 105 includes, among other things, implantable medical device 110 and lead system 108. In various embodiments, implantable medical device 110 is an implantable CRM device including one or more of a pacemaker, a cardioverter/defibrillator, a cardiac resynchronization therapy (CRT) device, a cardiac remodeling control therapy (RCT) device, a neurostimulator, a drug delivery device or a drug delivery controller, and a biological therapy device. As illustrated in FIG. 10, implantable medical device 110 is implanted in a body 102. In various embodiments, lead system 108 includes leads for sensing physiological signals and delivering pacing pulses, cardioversion/defibrillation shocks, neurostimulation pulses, pharmaceutical agents, biological agents, and/or other types of energy or substance for treating cardiac disorders. In one embodiment, lead system 108 includes one or more pacing-sensing leads each including at least one electrode placed in or on a heart 101 for sensing electrogram and/or delivering pacing pulses. In other embodiments, electrodes placed in body 102 but away from heart 101 are used to sense physiological signals and deliver pacing pulses, cardioversion/defibrillation shocks, neurostimulation pulses, pharmaceutical agents, biological agents, and/or other types of energy or substance for treating cardiac disorders. In a specific embodiment, one or more electrodes are incorporated onto implantable medical device 110 for subcutaneous placement.

Implantable medical device 110 includes a cardiac pacing system 120. Cardiac pacing system 120 is capable of delivering cardiac protection pacing therapies (CPPT) through lead system 108. The delivery of a cardiac protection pacing therapy is timed as a cardiac protection pacing sequence including alternating pacing and non-pacing periods. In one embodiment, in addition to the cardiac protection pacing therapy, cardiac pacing system 120 also delivers one or more other cardiac pacing therapies, such a bradycardia pacing therapy, CRT, and RCT. If another pacing therapy is being delivered when a cardiac protection pacing sequence is to be initiated, that pacing therapy is temporarily suspended to allow the delivery of the cardiac protection pacing therapy and resumed upon completion of the cardiac protection pacing sequence.

External system 115 allows a user such as a physician or other caregiver or a patient to control the operation of implantable medical device 110 and obtain information acquired by implantable medical device 110. In one embodiment, external system 115 includes a programmer communicating with implantable medical device 110 bi-directionally via telemetry link 112. In another embodiment, external system 115 is a patient management system including an external device communicating with a remote device through a telecommunication network. The external device is within the vicinity of implantable medical device 110 and communicates with implantable medical device 110 bi-directionally via telemetry link 112. The remote device allows the user to monitor and treat a patient from a distant location. The patient monitoring system is further discussed below, with reference to FIG. 13.

Telemetry link 112 provides for data transmission from implantable medical device 110 to external system 115. This includes, for example, transmitting real-time physiological data acquired by implantable medical device 110, extracting physiological data acquired by and stored in implantable medical device 110, extracting therapy history data stored in implantable medical device 110, and extracting data indicating an operational status of implantable medical device 110 (e.g., battery status and lead impedance). Telemetry link 112 also provides for data transmission from external system 115 to implantable medical device 110. This includes, for example, programming implantable medical device 110 to acquire physiological data, programming implantable medical device 110 to perform at least one self-diagnostic test (such as for a device operational status), and programming implantable medical device 110 to deliver at least one therapy.

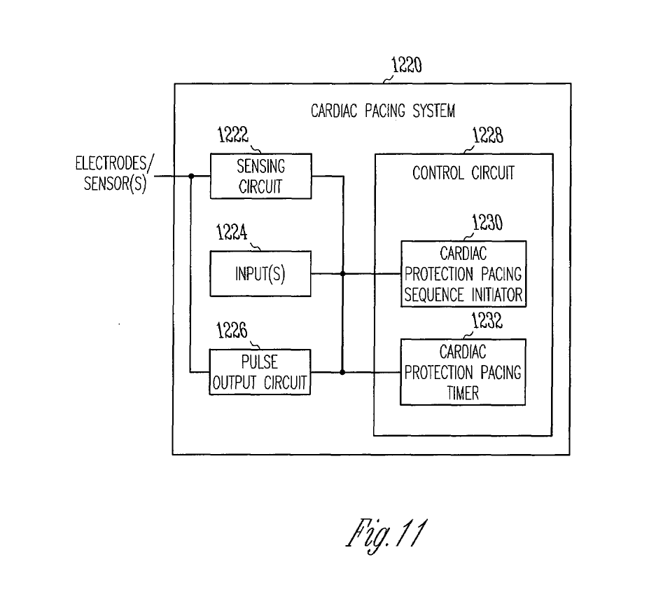

FIG. 11 is a block diagram illustrating an embodiment of portions of the circuit of a cardiac pacing system 1220. Cardiac pacing system 1220 is a specific embodiment of cardiac pacing system 120 and includes a sensing circuit 1222, IPT input(s) 1224, a pulse output circuit 1226, and a control circuit 1228. Sensing circuit 1222 senses one or more signals using a plurality of electrodes and/or one or more sensors. The one or more signals are indicative of cardiac parameters. IPT input(s) 1224 provide information regarding intrinsic AV intervals, interventricular (VV) timing, QRS width and LV lead/electrode location from the one or more signals and/or inputs. Pulse output circuit 1226 delivers pacing pulses to heart 101. Control circuit 1228 controls the delivery of the pacing pulses based on the one or more sensed signals and/or based on the one or more IPT inputs. In various embodiments, cardiac pacing system 1220 is substantially contained in an implantable housing of implantable medical device 110.

Control circuit 1228 includes a cardiac protection pacing sequence initiator 1230 and a cardiac protection pacing timer 1232. Cardiac protection pacing sequence initiator 1230 initiates one or more cardiac protection pacing sequences using parameters recommended based on the IPT input(s). The one or more cardiac protection pacing sequences each include alternating pacing and non-pacing periods. The pacing periods each have a pacing duration during which a plurality of pacing pulse is delivered. The non-pacing periods each have a non-pacing duration during which no pacing pulse is delivered. Once a cardiac protection pacing sequence is initiated, cardiac protection pacing timer 1232 times that sequence.

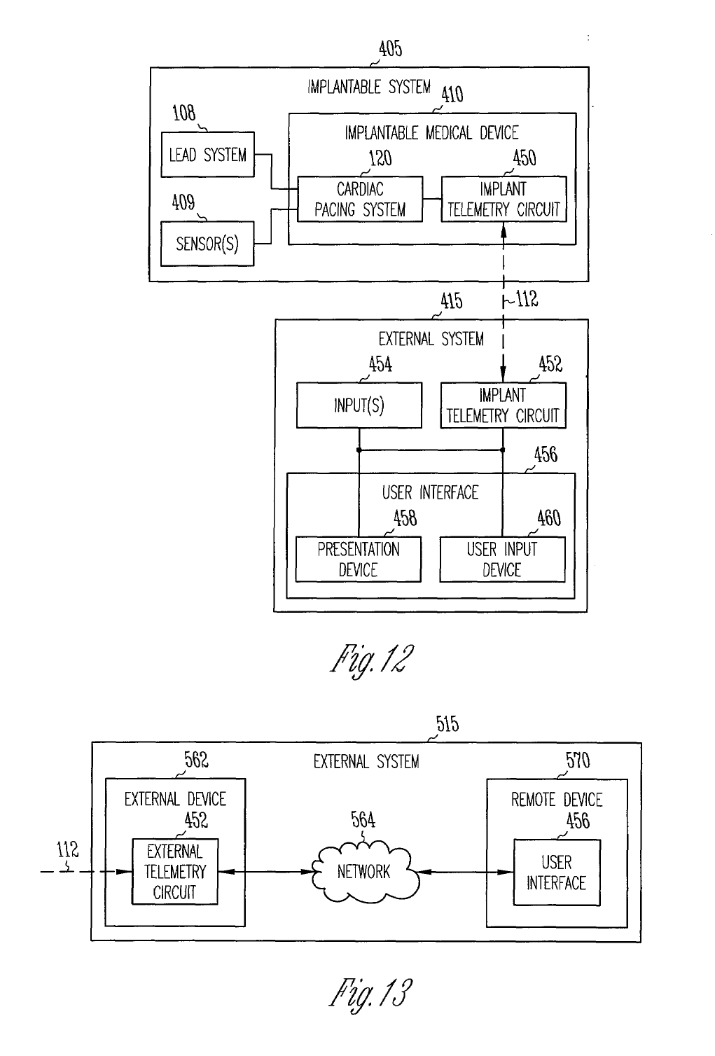

FIG. 12 is a block diagram illustrating an embodiment of portions of circuits of an implantable system 1405 and an external system 1415. Implantable system 1405 is a specific embodiment of implantable system 105. External system 1415 is a specific embodiment of external system 115.

Implantable system 1405 includes lead system 108, one or more sensors 1409, and implantable medical device 1410. Sensor(s) 1409 includes electrodes, accelerometer(s), pressure sensor(s), and/or other sensors for sensing one or more signals required for the operation of implantable medical device 1410, including detection of IPT input(s). In various embodiments, sensor(s) 1409 are included in an implantable housing of implantable medical device 1410, attached to implantable medical device 1410, coupled to implantable medical device 1410 through wired or wireless connections, and/or incorporated into lead system 108. Implantable medical device 1410 is a specific embodiment of implantable medical device 110 and includes cardiac pacing system 120 (including its various embodiments) and an implant telemetry circuit 1450.

External system 1415 includes an external telemetry circuit 1452, an IPT input(s) receiver 1454, and a user interface 1456. External telemetry circuit 1452 and implant telemetry circuit 1450 supports telemetry link 112, through which directional communication is performed between external system 1415 and implantable system 1405. User interface 1456 includes a presentation device 1458 and a user input device 1460. Presentation device 1458 includes a display screen. In one embodiment, presentation device 1458 further includes a printer and a speaker. User input device 1460 allows programming of implantable medical device 1410, including the entry of commands for initiating one or more cardiac protection pacing sequences and/or parameters controlling the delivery of the cardiac protection pacing therapy. In one embodiment, portions of presentation device 1458 and user input device 1460 are integrated as an interactive screen. IPT input(s) receiver 1454 receives sensed data regarding, for example, sensed intrinsic timing, lead/electrode location and AV intervals, via telemetry from the implantable system, according to various embodiments. In various embodiments, IPT input(s) may be entered by a user, such as a medical professional, using user input device 1460. In one embodiment, external system 1415 includes a programmer. In another embodiment, external system 1415 includes a patient management system as discussed below with reference to FIG. 13.

FIG. 13 is a block diagram illustrating an embodiment of an external system 1515, which is a specific embodiment of external system 1415. As illustrated in FIG. 5, external system 1515 is a patient management system including an external device 1562, a telecommunication network 1564, and a remote device 1570. External device 1562 is placed within the vicinity of an implantable medical device and includes external telemetry system 1452 to communicate with the implantable medical device via telemetry link 112. Remote device 1570 is in one or more remote locations and communicates with external device 1562 through network 1564, thus allowing a physician or other caregiver to monitor and treat a patient from a distant location and/or allowing access to various treatment resources from the one or more remote locations. In one embodiment, as illustrated in FIG. 5, remote device 1570 includes user interface 1456. This allows the user to initiate and/or adjust the cardiac protection pacing.

One of ordinary skill in the art will understand that, the modules and other circuitry shown and described herein can be implemented using software, hardware, and combinations of software and hardware. As such, the terms module and circuitry, for example, are intended to encompass software implementations, hardware implementations, and software and hardware implementations.

The methods illustrated in this disclosure are not intended to be exclusive of other methods within the scope of the present subject matter. Those of ordinary skill in the art will understand, upon reading and comprehending this disclosure, other methods within the scope of the present subject matter. The above-identified embodiments, and portions of the illustrated embodiments, are not necessarily mutually exclusive. These embodiments, or portions thereof, can be combined. In various embodiments, the methods are implemented using a computer data signal embodied in a carrier wave or propagated signal, that represents a sequence of instructions which, when executed by one or more processors cause the processor(s) to perform the respective method. In various embodiments, the methods are implemented as a set of instructions contained on a computer-accessible medium capable of directing a processor to perform the respective method. In various embodiments, the medium is a magnetic medium, an electronic medium, or an optical medium.

The above detailed description is intended to be illustrative, and not restrictive. Other embodiments will be apparent to those of skill in the art upon reading and understanding the above description. The scope of the invention should, therefore, be determined with reference to the appended claims, along with the full scope of equivalents to which such claims are entitled.

What is claimed is:

1. A method, comprising:
sensing a signal indicative of an incidence of angina and an angina region being a myocardial region affected by the angina;
detecting the incidence of angina and locating the angina region;
selecting a pacing location remote from the angina region; and
initiating cardiac protective pacing therapy (CPPT) at the pacing location adapted to create increased stress at the angina region, to promote mass-redistribution and angiogenesis at the angina region to treat the angina.

2. The method of claim 1, wherein sensing a signal to detect an incidence of angina includes using a device-based method.

3. The method of claim 1, wherein sensing a signal to detect an incidence of angina includes using an imaging-based method.

4. The method of claim 1, wherein initiating CPPT includes initiating continuous pacing therapy.

5. The method of claim 1, wherein initiating CPPT includes initiating intermittent pacing therapy.

6. The method of claim 5, wherein initiating intermittent pacing therapy includes delivering pacing therapy in regular on/off pacing cycles.

7. The method of claim 6, wherein initiating intermittent pacing therapy includes delivering pacing therapy with at least three cycles of on/off pacing.

8. The method of claim 7, wherein initiating intermittent pacing therapy includes delivering pacing therapy with approximately 60 minutes of on/off pacing cycles.

9. The method of claim 1, wherein initiating CPPT includes using ST elevation to cause mild ischemia, and to change the pacing mode and/or duration of pacing.

10. The method of claim 1, further comprising:
monitoring patient activity level; and
using the monitored level as an index of therapy efficacy in a closed loop system.

11. A method, comprising:
delivering cardiac protective pacing therapy (CPPT) to provide a cardiac conditioning therapy to improve neural balance;
monitoring neural balance and a parameter indicative of a selected co-morbidity related to neural imbalance; and
titrating the CPPT based on the monitored balance and parameter.

12. The method of claim 11, wherein monitoring a parameter indicative of a selected co-morbidity includes allowing a physician to select the co-morbidity.

13. The method of claim 11, wherein monitoring a parameter indicative of a selected co-morbidity includes monitoring a parameter of interest for sleep disordered breathing.

14. The method of claim 11, wherein monitoring a parameter indicative of a selected co-morbidity includes monitoring a parameter of interest for apnea.

15. The method of claim 11, wherein monitoring a parameter indicative of a selected co-morbidity includes monitoring a parameter of interest for atrial fibrillation (AF) burden.

16. The method of claim 11, wherein monitoring a parameter indicative of a selected co-morbidity includes monitoring a parameter of interest for hypertension.

17. The method of claim 11, wherein monitoring a parameter indicative of a selected co-morbidity includes monitoring a parameter of interest for renal dysfunction.

18. The method of claim 11, wherein monitoring a parameter indicative of a selected co-morbidity includes monitoring apnea-hypopnea index (AHI).

19. The method of claim 11, wherein titrating the CPPT includes continuing CPPT unchanged if the sensed parameter improves.

20. The method of claim 11, wherein titrating the CPPT includes discontinuing CPPT unchanged if the sensed parameter degrades or is unchanged.

21. The method of claim 11, wherein titrating the CPPT includes continuing CPPT with a new therapy mode if the sensed parameter degrades or is unchanged.

22. A medical device for use in a body, comprising:
a sensing circuit to receive sensed signal indicative of an incidence of angina and an angina region being a myocardial region affected by the angina;
a pacemaker circuit adapted to deliver an electrical signal through at least one electrode to a pacing location remote from the angina region; and
a controller to communicate with the sensing circuit and to control the pacemaker circuit to provide CPPT to the pacing location adapted to create increased stress at the angina region, to promote mass-redistribution and angiogenesis at the angina region to treat the angina.

23. The device of claim 22, wherein the device includes an implantable cardiac rhythm management (CRM) pulse generator (PG) with intra-cardiac leads.

24. A medical device for use in a body, comprising:
a sensing circuit adapted to receive sensed signals indicative of neural balance and adapted to receive sensed parameters indicative of a selected co-morbidity related to neural imbalance;
a pacemaker circuit adapted to deliver an electrical signal through at least one electrode to a target location; and
a controller adapted to communicate with the sensing circuit and to control the pacemaker circuit to provide CPPT to the target location, and farther adapted to titrate the CPPT based on the sensed signals and parameters.

25. The device of claim 24, wherein the selected co-morbidity includes sleep disordered breathing.
